(19) United States
(12) Patent Application Publication
(10) Pub. No.: US 2024/0081731 A1
(43) Pub. Date: Mar. 14, 2024

(54) METHOD FOR EVALUATING SLEEP AND SYSTEM FOR SAME

(71) Applicant: Sawylan Technology LLC, Plano, TX (US)

(72) Inventor: Yongling Ji, Plano, TX (US)

(21) Appl. No.: 17/944,008

(22) Filed: Sep. 13, 2022

(51) Int. Cl. A61B 5/00; A61B 5/0205

(52) U.S. Cl. CPC A61B 5/4815; A61B 5/0205; A61B 5/7264

(57) ABSTRACT

A method for evaluating a sleep quality is provided. The method includes the steps of acquiring a respiratory signal and a heart rate signal, capturing more than two consecutive windows of equal time length from the respiratory signal and the heart rate signal respectively, and performing a cross-spectrum analysis on a time-aligned respiratory window and a heart rate window, and obtaining a cross-spectrum amplitude matrix of the consecutive windows, constructing a stability coefficient matrix according to a cosine similarity between the cross-spectral amplitude matrix of the consecutive windows and estimating the sleep quality according to the stability coefficient matrix.

### FIELD

The invention relates to an improved sleep evaluation method and system. Specifically, the invention relates to a method for evaluating sleep and a system thereof.

### BACKGROUND

Sleep quality evaluation plays a key role in the diagnosis and treatment of sleep related disorders. Currently, the methods for evaluating sleep quality commonly used by doctors include subjective scale and polysomnography monitoring. Polysomnography monitoring requires multiple electrodes to be attached to patients for collecting electroencephalogram (EEG), electro-oculogram (OEG), and electromyography (EMG) signals. Polysomnography monitoring identifies sleep quality according to these electrical signals. The characteristics of the monitored EEG waves are mainly relied on for the evaluation. However, in practical application, polysomnography is complicated and must be operated in professional sleep laboratories. The electrodes attached to the head undoubtedly affect the sleep of patients, resulting in what is known as a first night effect. A such, the results of polysomnography often cannot accurately determine the sleep quality of patients.

With increased attention paid to promoting healthy sleeping habits, there are a growing number of portable sleep evaluation products on the market. In order to avoid the discomfort caused by the process of collecting EEG signals, these portable sleep monitoring devices usually do not collect EEG signals but instead utilize other methods to evaluate the sleep quality of a patient. Such a method is disclosed in Chinese Patent No. CN 104095615B wherein the activity state of the patient's body is determined by recording a related change in motion signals so as to infer the sleep state, or the sleep state is identified according to the change in the observed heart rate variability index by recording the electrocardiogram signal. However, such sleep determination methods by a single signal are not accurate.

### SUMMARY

A sleep evaluation method and system are disclosed herein, aiming to solve the problem that signal acquisition is complex and leads to discomfort, or sleep evaluation based on a singular signal leads to inaccurate results.

The sleep evaluation method in the disclosure includes the steps as follows.

A respiratory signal and a heart rate signal are acquired.

Two or more consecutive windows of equal time length are intercepted from the respiration signal and the heart rate signal respectively. A cross spectrum analysis is then performed on the respiratory window and the heart rate window aligned in time to obtain the cross spectrum amplitude matrix of the consecutive windows.

A stabilization coefficient matrix is constructed according to cosine distances between cross spectral amplitude matrices between the consecutive windows.

The sleep quality is evaluated according to the stabilization coefficient matrix.

Further, the amplitudes adjacent to the sampling point in the amplitude matrix are used to form eigenvectors. The cosine distance of the eigenvectors at the same frequency point in the consecutive window is calculated, and a stabilization coefficient matrix is constructed between the consecutive windows by the cosine distance.

Further, the stabilization coefficient matrix is obtained by a cosine distance weighted average of consecutive windows.

Further, weighted averaging is performed on the cross-spectrum amplitude matrix of the consecutive windows to obtain an average amplitude matrix. The product of the stability coefficient matrix and the reconstructed cross-spectrum amplitude matrix is calculated. A sleep quality is evaluated based on the product.

Further, adjacent ones of the consecutive windows include overlapping portions. Adjacent ones of the consecutive windows are overlapped by at least 50%.

Further, the eigenvector is a vector composed of amplitudes of a plurality of consecutive frequency points, and preferably, the vector is composed of amplitudes of three consecutive frequency points.

Furthermore, the cross-spectrum amplitude matrix is filled with frequency points at the beginning and the end, and the filled amplitude is equal to the amplitude of the beginning and the end respectively.

A sleep evaluation system is disclosed, which includes a signal acquisition module, an analysis module and an output module.

The signal obtaining module is configured to obtain a respiratory signal and a heart rate signal of a user.

The analysis module is configured to perform characteristic processing on the respiratory signal and the heart rate signal to obtain a stability coefficient matrix.

The output module outputs a result of identifying the sleep state according to the stabilization coefficient matrix obtained by the analysis module.

Further, the analysis module may further process the respiratory signal and the heart rate signal to obtain a sleep index, and the output module determines whether the user is in a deep sleep or a light sleep according to the sleep index.

Another aspect of the disclosure also provides a storage medium having stored thereon a computer program that, when executed by a processor, executes steps in the sleep evaluation method disclosed herein.

The disclosure provides a sleep evaluation method, in which a stability coefficient matrix is obtained by analyzing a heart rate signal and a respiratory signal to identify a sleep state. The index of sleep degree is obtained according to the stability coefficient matrix and the cross spectrum of heart rate signal and respiration signal. The sleep evaluation method and system provided by the disclosure do not need to collect the electroencephalogram, which does not cause discomfort to the user and does not affect the sleep of the user. Meanwhile, the method retains more sleep-related characteristics, and the interaction between respiration signal and heart rate signal in sleep is analyzed, resulting in a more accurate of the recognition.

### BRIEF DESCRIPTION OF THE DRAWINGS

FIG. 1 is a flowchart of a sleep evaluation method according to an embodiment of the disclosure.

FIG. 2 is a data flow diagram of a sleep evaluation method according to an embodiment of the disclosure.

FIG. 3 is a schematic diagram of an intercepted window in an embodiment of the disclosure.

FIG. 4 is a schematic diagram of a filling matrix in an embodiment of the disclosure.

FIG. 5 is a flowchart of obtaining a stability coefficient matrix according to an embodiment of the disclosure.

FIG. 6 is a data flow diagram of FIG. 5.

FIG. 7 is a schematic diagram of eigenvector extraction in an embodiment of the disclosure.

FIG. 8 is a flowchart of obtaining a sleep index in an embodiment of the disclosure.

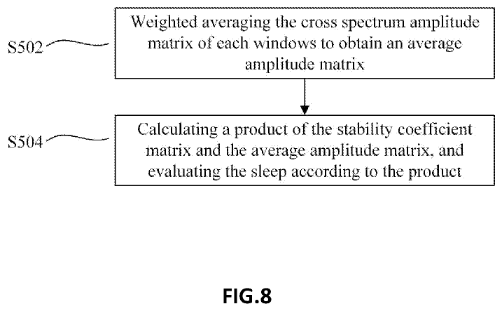

FIG. 9 is a sleep spectrum obtained in an embodiment of the disclosure.

FIG. 10 is a system for evaluating sleep according to an embodiment of the disclosure.

### DETAILED DESCRIPTION

A sleep evaluation method and apparatus provided by the disclosure are described below in conjunction with the drawings in the embodiments of the disclosure. In the following description, for the purposes of full description and explanation, specific details are set forth in order to provide a more thorough understanding, without limiting the scope of protection of the disclosure.

### Embodiment 1

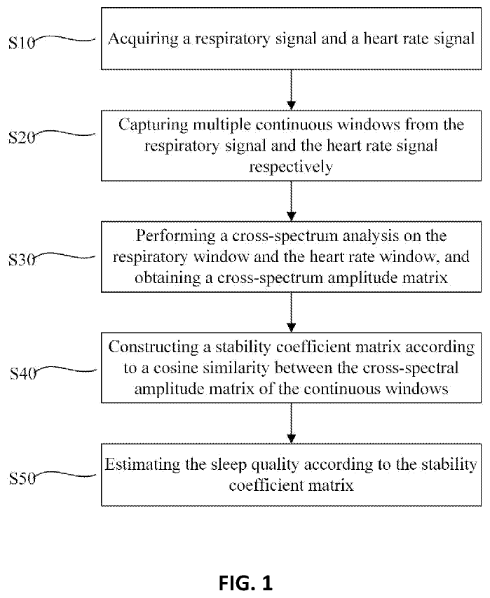

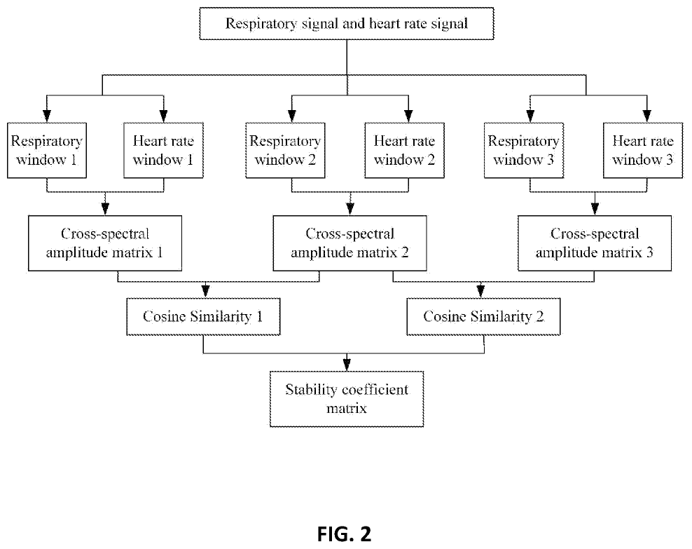

In an embodiment of the disclosure, a sleep evaluation method is provided. FIG. 1 is a flowchart of an embodiment of the disclosure, and FIG. 2 is a data flow diagram of performing the steps of FIG. 1. As shown in the FIG. 1, the sleep evaluation method of this embodiment includes the steps as follows.

In step S10, a respiratory signal and a heart rate signal are acquired. The respiration signal and the heart rate signal may be real-time acquired signals, or may be whole-segment signals when the whole sleep monitoring is completed. The sampling frequency of the respiratory signal and the heart rate signal is the same. In this embodiment, a sampling frequency of 2 Hz can be selected. There are many ways to acquire the heart rate signal, for example, QRS wave is acquired by the ECG electrode. The heartbeat interval is acquired by the R wave position, The heart rate signal is indicated by the heartbeat intervals, and the heart rate signal can also be indicated by the pulse signal at the wrist. The respiratory signal can be obtained by chest impedance signal, mouth and nose airflow signal, chest and abdomen movement signal.

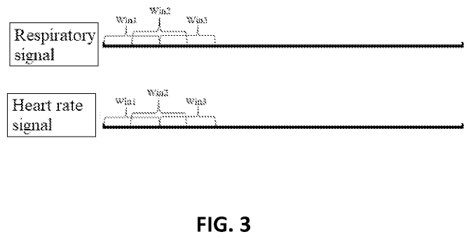

In step S20, multiple windows are respectively intercepted for the respiratory signal and the heart rate signal, and at least two consecutive windows of equal time length are respectively intercepted from the respiratory signal and the heart rate signal. It should be ensured that every respiratory window intercepted has a heart rate window aligned with its time. In this embodiment, the number of intercepting windows is set to 3. FIG. 3 shows a manner of intercepting windows in this embodiment, and Win1, Win2, and Win3 are windows of equal length. The overlapping of adjacent Win 1 and Win2 in the respiratory signal is 50%. Similarly, Win2 and Win3 are of 50% data overlapping. Accordingly, the 3-segment window corresponding to the time in the respiratory signal is also intercepted from the heart rate signal. In other embodiment, it should be understood that that intent of the disclosure can be achieved by intercepting at least two windows. It should also be understood that 50 percent data overlapping between adjacent windows in this embodiment is only one preferred mode of the disclosure, while in other embodiments, data overlapping may be set to other ranges.

As shown in FIG. 2, after the step S20, three sets of time-aligned windows are obtained. Respiratory window 1 and heart rate window 1 form one set. The respiratory window 2 and heart rate window 2 form another set. The respiratory window 3 is grouped with the heart rate window 3. The respiratory window 1 and the respiratory window 2 are adjacent windows, and the respiratory window 2 and the respiratory window 3 are adjacent windows. Similarly, the heart rate window corresponding to the respiratory window is the same.

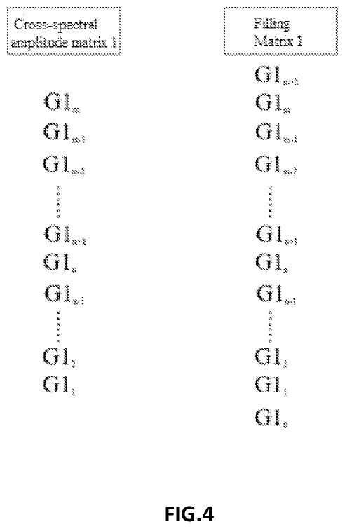

In step S30, the cross spectrum of the respiratory window and the heart rate window is respectively calculated to obtain the cross spectrum amplitude matrix. The two windows are firstly transformed from time domain to frequency domain, then the cross spectrum is calculated. Finally, the cross spectrum is multiplied by its conjugate to obtain the cross spectrum amplitude matrix. In this embodiment, three cross-spectrum amplitude matrices can be obtained from three sets of corresponding respiration and heart rate window. The following description will be given with respiration window 1 and heart rate window 1. The respiratory window 1 and the heart rate window 1 perform step S30 to obtain the cross-spectrum amplitude matrix 1, such as 4a in FIG. 4. The cross-spectrum amplitude matrix 1 contains the values of m frequency points, and Gln represents the amplitude at the n-th frequency point. Similarly, the number of frequency points in the cross-spectrum amplitude matrices 2 and 3 is also m. The cross-spectrum amplitude matrix is determined to be adjacent to the respiratory window or the heart rate window by calculating the cross-spectrum. Therefore, the cross-spectrum amplitude matrix 1 is adjacent to the cross-spectrum amplitude matrix 2. The cross-spectrum amplitude matrix 3 is also adjacent to the cross-spectrum amplitude matrix 2.

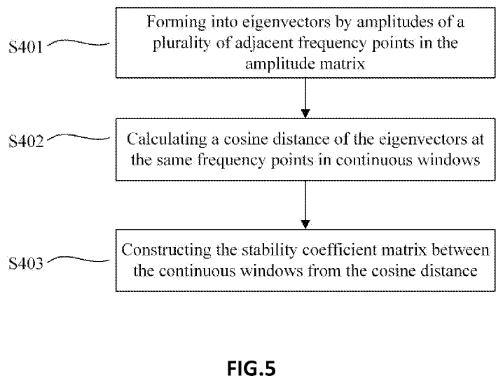

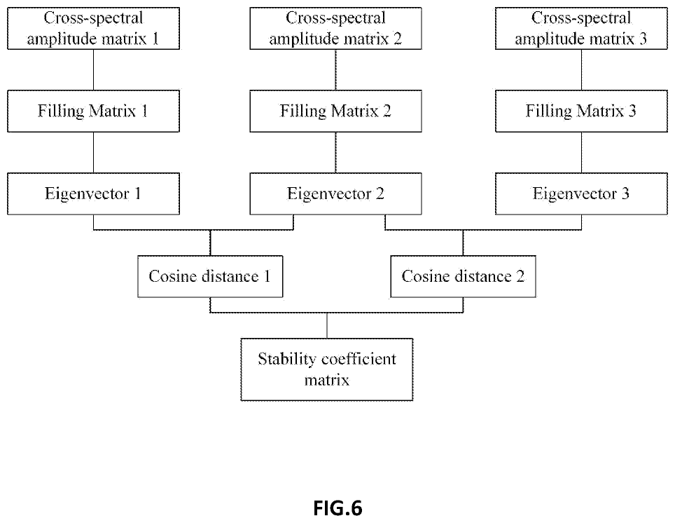

In step S40, an eigenvector is selected from the cross-spectrum amplitude matrix, and a cosine distance of the eigenvector is calculated to obtain a stabilization coefficient matrix. The cross-spectrum amplitude matrix includes a plurality of frequency points, and the amplitudes of two or more frequency points are selected from the same cross-spectrum amplitude matrix to form an eigenvector, and accordingly, other cross-spectrum matrices also select the amplitude at the same frequency to form eigenvectors. The Cosine distance of eigenvectors of adjacent crossed spectral matrices is calculated. By selecting amplitude values at different frequency points to form different eigenvectors, a group of adjacent cross-spectrum matrices can obtain a sequence containing multiple cosine distances. When there are multiple groups of adjacent cross-spectrum matrices, weights are assigned to a plurality of sequences composed of cosine distances, and the average value is calculated to obtain a stabilization coefficient matrix. In other embodiment, when only two windows of the respiratory signal and the heart rate signal are intercepted in step S20, there is only one set of adjacent cross-spectrum matrices, the sequence composed of multiple cosine distances obtained from the group of adjacent cross-spectrum matrices is the stabilization coefficient matrix. In this embodiment, three consecutive frequency points are selected in turn to form eigenvectors. As shown in FIGS. 5 and 6, the implementation of step S40 further includes the steps as follows.

S401: the cross spectrum amplitude matrix is filled according to the dimension of the eigenvectors. As shown in FIG. 4, FIG. 4a is the cross-spectrum amplitude matrix 1, and FIG. 4b is the filling matrix 1. Because the dimension of the eigenvector in this embodiment is 3, one frequency point is added at the beginning and the end of the cross spectrum amplitude moment 1, respectively, and the filling matrix 1 contains (m+2) frequency points in total. Every 3 consecutive frequency points form an eigenvector, then the filling matrix 1 generates a total of m eigenvectors. The amplitudes of G11 and G1m are assigned to G10 and G1(m+1), respectively. Similarly, the cross-spectrum amplitude matrix 2 and the cross-spectrum amplitude matrix 3 are filled to obtain a filling matrix 2 and a filling matrix 3.

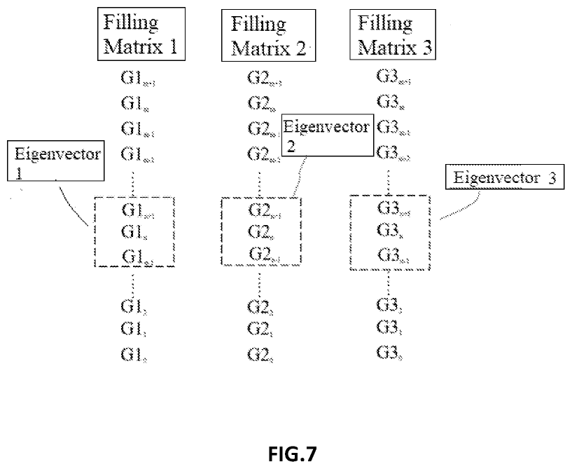

S402: The cosine distances of the same frequency point eigenvector of the cross spectrum amplitude matrix of the adjacent windows are calculated. As shown in FIG. 7, the nth eigenvector in the filled matrix is composed of the nth frequency point and its preceding and succeeding adjacent frequency points. In the filled matrix 1, the nth eigenvector 1 is [G1n−1 G1n G1n+1], and in the filled matrix 2 and the filled matrix 3, the nth eigenvector 2 is [G2n−1 G1n G2n+1]. The nth eigenvector 3 is [G3n−1 G3n G3n+1]. The cosine distance 1 of the nth eigenvector 1 and the eigenvector 2 can be calculated by the following formula:

$${CosDistance}_{1} = \frac{G {1}_{n - 1} \cdot G {2}_{n - 1} + G {1}_{𝔫} \cdot G {2}_{n} + G {1}_{n + 1} \cdot G {2}_{n + 1}}{\sqrt{G {1}_{n - 1}^{2} + G {1}_{n}^{2} + G {1}_{n + 1}^{2}} \cdot \sqrt{G {2}_{𝔫 - 1}^{2} + G {2}_{n}^{2} + G {2}_{n + 1}^{2}}}$$

Similarly, the cosine distance 2 of the nth eigenvector 2 and the eigenvector 3 is calculated as follows:

$$CosDist {ance}_{2} = \frac{G {3}_{n - 1} \cdot G {2}_{n - 1} + G {3}_{𝔫} \cdot G {2}_{n} + G {3}_{n + 1} \cdot G {2}_{n + 1}}{\sqrt{G {3}_{n - 1}^{2} + G {3}_{n}^{2} + G {3}_{n + 1}^{2}} \cdot \sqrt{G {2}_{𝔫 - 1}^{2} + G {2}_{n}^{2} + G {2}_{n + 1}^{2}}}$$

S403: obtain a stabilization coefficient matrix by weighted averaging cosine distances between a plurality of windows. In this embodiment, two cosine distances are included at the nth frequency point: cosine distance 1 and cosine distance 2. Weighted averaging is performed on the cosine distance 1 and the cosine distance 2. The weight of the cosine distance 1 is w and the weight of the cosine distance 2 is 1-w, then the stability coefficient Coef can be obtained as follows:

Coef=w·CosDistance₁+(1−w)·CosDistance₂

In the filling matrices 1-3, when n is sequentially taken from 1-m, a stabilization coefficient matrix CoefMat correspond to the frequency number m of the cross-spectral amplitude matrix can be obtained.

Step S50: the sleep state is evaluated according to the stability coefficient matrix. Windows are sequentially taken from the respiratory signal and the heart rate signal to complete the analysis of the whole sleep period, and the sleep state is evaluated according to the stability coefficient matrix. Each time period corresponds to a stability coefficient matrix, the cosine distance constituting the stability coefficient matrix is averaged, and whether the user is in a sleep state is judged according to the averaged value, when the averaged value is in the range of 0.5 to 1, the user is considered to be asleep.

In this embodiment, the step S50 further includes the steps as follows.

S502: an average amplitude matrix of the cross-spectrum amplitude matrices of the plurality of windows is calculated. Specifically, after performing step S30, a plurality of cross-spectrum amplitude matrices are obtained, and a weighted average of the plurality of cross-spectrum amplitude matrices is obtained to obtain an average amplitude matrix. In this embodiment, the three weight coefficients f1, f2, and f3 are respectively assigned to the three cross-spectrum amplitude matrices to obtain the average amplitude matrix.

S504: the product of the average amplitude matrix and the stability coefficient matrix are taken as indexes for evaluating sleep. The three-segment windows intercepted in this embodiment is described in the following, where G 1, G2 and G3 respectively represent the three cross-spectrum amplitude matrices in FIG. 2, and the stability coefficient matrix obtained in step S40 is represented by CoefMat, multiplied by the average amplitude matrix obtained in S501, the sleep index SC is calculated as follows:

SC=(G1·f1+G2·f2+G3·f3)·CoefMat

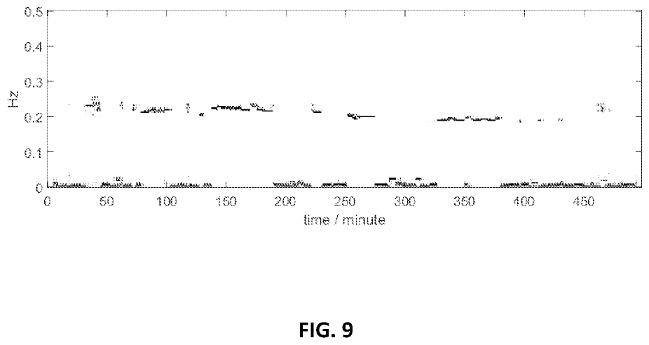

Sleep evaluation is performed according to the spectrum of sleep index SC, as shown in FIG. 9, which is a SC spectrum of a sleep period, when the sleep index is concentrated in the range of 0.15 Hz to 0.4 Hz in the SC spectrum, the user is considered to be in a stable sleep state. If the sleep index spectrum is in the range of 0.2 Hz˜0.3 Hz within 150 minutes˜180 minutes, the user is considered to be in a deep sleep state. If the SC spectrum is in a low frequency range, the user is considered to be in a light sleep state.

In other embodiments, the calculation of the sleep index may be performed after step S401 in addition to steps S502 and S504. In step S403, the mean eigenvector is obtain by weighted average the eigenvectors composed of the same frequency points in the different cross-spectrum amplitude matrix, and the mean eigenvector is multiplied by the stability coefficient Coef, N is sequentially taken from 1 to m to obtain a sleep index.

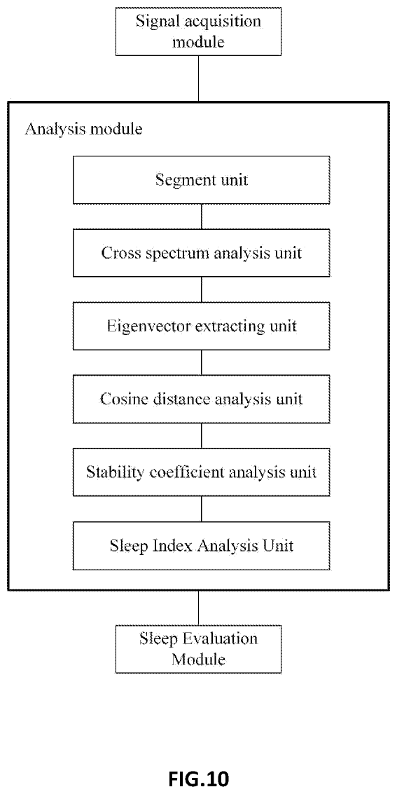

As shown in FIG. 10, a sleep evaluation system is also provided, which includes a signal acquisition module, an analysis module, and a sleep evaluation module.

The signal acquisition module is configured to acquire a respiration signal and a heart rate signal. Heart rate signal acquisition is to extract signal representing heart rate and heartbeat interval from collected signal, for example, heart rate signal is extracted from ECG signal and pulse wave signal. Respiratory signal acquisition is to extract signals representing respiratory frequency from acquired signals, for example, respiratory signals are extracted from thoracic impedance signals, oro-nasal airflow signals, and thoracic-abdominal motion signals. It should be understood that the signal acquisition module may also directly import the respiratory signal and the heart rate signal.

The analysis module analyzes the heart rate signal and the respiratory signal extracted by the heart rate signal acquisition module and the respiratory signal acquisition module. The analysis module includes a plurality of analysis units.

The segmentation unit is configured to intercept a plurality of consecutive windows of the respiratory signal and the heart rate signal to obtain the respiratory window and the heart rate window. The number of windows and the overlapping data range of adjacent windows can be set.

A cross spectrum analysis unit is configured to obtain cross spectrum amplitude matrices of a respiratory window and a heart rate window, and further configured to obtain an average amplitude matrix of a multi-segment window.

An eigenvector extracting unit is configured to extract an eigenvector from the cross-spectrum amplitude matrix. The eigenvector dimension and a frequency formed by the eigenvector can be set.

A cosine distance analysis unit is configured to calculate a cosine distance between eigenvectors from adjacent cross spectral amplitude matrixes.

A stability coefficient analysis unit is configured to construct a stability coefficient matrix according to a plurality of cosine distances. The weight coefficients belonging to different adjacent cross-spectrum amplitude matrices can be set.

A sleep index analysis unit is configured to obtain a sleep index according to a product of the stability coefficient matrix obtained by the stability coefficient analysis unit and the average amplitude matrix obtained by the cross spectrum analysis unit.

The output module is configured to output a sleep result, judge whether to enter sleep according to the stability coefficient matrix, obtain a sleep spectrum chart according to the sleep index, and judge whether the user is in a deep sleep or a light sleep.

Embodiments of the disclosure also provide a storage medium on which a computer program is stored. When the computer program is executed by a processor, the steps of any of the above embodiments may be performed. The media may be implemented by any type of storage device, including non-volatile and/or volatile memory. The non-volatile memory includes a read only memory (ROM), a programmable ROM (PROM), an electrically programmable ROM (EPROM), an electrically erasable programmable ROM (EEPROM), or a flash memory. The volatile memory may include random access memory (RAM) or an external cache memory. By way of illustration and not limitation, RAM is available in various forms such as static RAM, dynamic RAM, synchronous DRAM, dual data rate SDRAM, enhanced SDRAM, synchronous link DRAM, and the like.

The above embodiments only describe and present the disclosure, and the disclosure is not limited to the scope of the above disclosed embodiments, and any modification included in the scope of the claims or equivalent belongs to the scope of protection of the disclosure.

What is claimed is:

1. A method for evaluating sleep quality, comprising:
acquiring a respiratory signal and a heart rate signal;
capturing more than two consecutive windows of equal time length from the respiratory signal and the heart rate signal respectively, and performing a cross-spectrum analysis on a time-aligned respiratory window and a heart rate window, and obtaining a cross-spectrum amplitude matrix of the consecutive windows;
constructing a stability coefficient matrix according to a cosine similarity between the cross-spectral amplitude matrix of the consecutive windows; and
estimating the sleep quality according to the stability coefficient matrix.

2. The method of claim 1, further comprising:
forming into eigenvectors by amplitudes of a plurality of adjacent frequency points in the amplitude matrix;
calculating a cosine distance of the eigenvectors at the same frequency points in consecutive windows; and
constructing the stability coefficient matrix between the consecutive windows from the cosine distance.

3. The method of claim 2, wherein the stability coefficient matrix is obtained by weighted average of the cosine distance of the consecutive windows.

4. The method of claim 1, further comprising:
weighted averaging the cross spectrum amplitude matrix of each windows to obtain an average amplitude matrix; and
calculating a product of the stability coefficient matrix and the average amplitude matrix, and evaluating the sleep according to the product.

5. The method of claim 1, wherein adjacent windows in the consecutive windows include overlapping portions.

6. The method of claim 2, wherein the eigenvectors are composed of amplitudes of a plurality of continuous frequency points.

7. The method of claim 6, wherein the amplitude matrix is filled with frequency points at a beginning and an end.

8. A system for evaluating a sleep quality, comprising a signal acquisition module, an analysis module and an output module; wherein,
the signal acquisition module is configured to acquiring a respiratory signal and a heart rate signal of a user;
the analysis module is configured to perform feature processing on the respiratory signal and the heart rate signal to obtain a stability coefficient matrix; and
the output module is configured to output a result of an identification of the sleep quality according to the stability coefficient matrix obtained by the analysis module.

9. A non-transitory storage medium having stored thereon a computer program, wherein the computer program, when executed by a processor, implements the steps of the method of claim 1.
